(19) United States
(12) Patent Application Publication
(10) Pub. No.: US 2003/0114414 A1
(43) Pub. Date: Jun. 19, 2003

(54) Methods for synthesizing substituted pyrimidines

(76) Inventors: Chengxin Zhi, Worcester, MA (US); Zheng-Yu Long, Worcester, MA (US); Andrzej Manikowski, Worcester, MA (US); George E. Wright, Worcester, MA (US)

Correspondence Address: FISH & RICHARDSON PC, 225 FRANKLIN ST, BOSTON, MA, 02110, US

(21) Appl. No.: 10/174,250

(22) Filed: Jun. 17, 2002

Related U.S. Application Data
(60) Provisional application No. 60/298,743, filed on Jun. 15, 2001.

(51) Int. Cl. C07H 19/067; A61K 31/7072; A61K 31/513

(52) U.S. Cl. 514/49; 514/269; 544/314; 536/28.5

(57) ABSTRACT

Methods of preparing N3-substituted-4-pyrimidones are disclosed. The methods include combining a 4-pyrimidone and a non-aqueous base, followed by an alkylating agent, for a time sufficient for the pyrimidone and the alkylating agent to react. Methods of preparing an N3-substituted-6-(substituted amino)uracil are also disclosed. The methods include (a) combining an N3-substituted-2-alkoxy-6-amino-4-pyrimidone with an amine compound selected from the group consisting of an amine salt and the corresponding free amine, to form a reaction mixture; and (b) heating the reaction mixture to at least 80° C. for a time sufficient for the N3-substituted-2-alkoxy-6-amino-4-pyrimidone and the amine compound to react to form the final product.

### CROSS-REFERENCE TO RELATED APPLICATION

This application claims benefit of priority from U.S. Provisional Patent Application No. 60/298,743, filed on Jun. 15, 2001, which is incorporated herein by reference in its entirety.

### STATEMENT AS TO FEDERALLY SPONSORED RESEARCH

The invention described herein was supported in whole or in part by SBIR grant number A141260 from the National Institutes of Health. The government thus has certain rights in the invention.

### TECHNICAL FIELD

This invention relates to methods for synthesizing substituted pyrimidines, and more particularly to methods for synthesizing certain N3-substituted pyrimidines.

### BACKGROUND

6-Aminopyrimidines, such as uracils, cytosines, and isocytosines, are valuable as therapeutic drugs. For example, suitably substituted 6-anilinopyrimidines are DNA polymerase inhibitors with antibacterial or cytotoxic activity. 6-Aminopyrimidines are also useful as intermediates in the preparation of bicyclic compounds, such as purines, pyrazolopyrimidines, xanthines, and related compounds. These bicyclic compounds can in turn be useful as therapeutic drugs. For example, suitably substituted 2-anilinopurines are DNA polymerase inhibitors with antibacterial or cytotoxic activity, or are helicase-primase inhibitors with antiviral activity. In addition, 1,9-dialkylxanthine analogs are useful as analogs of adenosine receptor antagonists.

It is often desirable for other positions of the 6-aminopyrimidines to be substituted. For example, N3-substituted-6-anilinopyrimidines are useful antibacterial compounds. However, when certain N3-substituted pyrimidines are allowed to react with aniline or aniline derivatives to form N3-substituted-6-anilinopyrimidines, the functional groups at the N3-position can be disrupted.

### SUMMARY

The invention provides new methods of synthesizing substituted pyrimidines. The invention is based on the discovery that one can synthesize 6-aminopyrimidines without disturbing functional groups at other positions. The invention is also based on the discovery of new methods for selectively substituting the N3-position of 4-pyrimidones, in preference to the O4-position of the pyrimidones. The new synthetic methods are useful in making a variety of therapeutic agents and provide the desired compounds in high yields.

In one aspect, the invention features a method of preparing an N3-substituted-4-pyrimidone. The method includes: (a) contacting a 4-pyrimidone with a non-aqueous base and an alkylating agent that includes an appropriately substituted alkyl moiety and a leaving group to form a reaction mixture; and (b) maintaining the reaction mixture for a time and under conditions sufficient for the 4-pyrimidone to be substituted in the N3-position with the appropriately substituted alkyl moiety.

The 4-pyrimidone can be a 2-alkoxy-6-amino-4-pyrimidone, e.g., a 2-methoxy-6-amino-4-pyrimidone. The non-aqueous base can be an alkali metal hydride, e.g., sodium hydride. The reaction mixture can include an alkali metal halide, e.g., lithium bromide, and/or an aprotic polar organic solvent, e.g., N,N-dimethylformamide.

In another aspect, the invention features a method of preparing a 6-(substituted amino)uracil. The method includes: (a) combining a 2-alkoxy-6-amino-4-pyrimidone with an amine compound selected from the group consisting of an amine salt and the corresponding free amine, to form a reaction mixture; and (b) heating the reaction mixture to at least 80° C. for a time sufficient for the 2-alkoxy-6-amino-4-pyrimidone and the amine compound to react to form the 6-(substituted amino)uracil.

The 6-(substituted amino)uracil can be an N3-substituted-6-(substituted amino)uracil, in which the N3-substituent can be an optionally substituted alkyl. The amine compound can be a free amine or a salt of that amine. When the amine compound is a free amine, step (a) can also include adding a salt of that amine to the reaction mixture. Thus, the reaction mixture can consist essentially of an N3-substituted-2-methoxy-6-amino-4-pyrimidone, the amine salt, and the corresponding free amine.

The amine salt can be an aryl amine salt, e.g., an optionally substituted aniline salt, such as a 3,4-disubstituted aniline salt, or a salt of 3-methyl-4-ethyl aniline. Alternatively, the amine salt can be a benzylamine salt, e.g., an optionally substituted benzylamine salt, such as a 3,4-disubstituted benzylamine salt, or a salt of 3,4-dichlorobenzylamine.

In another aspect, the invention features a method for preparing a 6-(substituted amino)uracil. The method includes: (a) contacting a 2-alkoxy-6-amino-4-pyrimidone with a non-aqueous base and an alkylating agent that includes an appropriately substituted alkyl moiety and a leaving group, for a time sufficient to produce an N3-substituted-2-alkoxy-6-amino-4-pyrimidone; (b) isolating the N3-substituted-2-alkoxy-6-amino-4-pyrimidone; (c) combining the N3-substituted-2-alkoxy-6-amino-4-pyrimidone with an amine compound selected from the group consisting of an amine salt and the corresponding free amine, to form a reaction mixture; and (d) heating the reaction mixture to at least 80° C. for a time sufficient for the N3-substituted-2-alkoxy-6-amino-4-pyrimidone and the amine compound to react to form the 6-(substituted amino)uracil.

The 2-alkoxy-6-amino-4-pyrimidone can be a 2-methoxy-6-amino-4-pyrimidone. The non-aqueous base can be an alkali metal hydride, e.g., sodium hydride. Step (a) can be conducted in the presence of an alkali metal halide, e.g., lithium bromide. Step (a) can also be conducted in an aprotic polar organic solvent, e.g., N,N-dimethylformamide.

The 6-(substituted amino)uracil can be an N3-substituted-6-(substituted amino)uracil, in which the N3-substituent can be an optionally substituted alky. The amine compound can be a free amine, and the reaction mixture can include or consist essentially of an N3-substituted-2-methoxy-6-amino-4-pyrimidone and a free amine. Alternatively, the amine compound can be an amine salt, and the reaction mixture can include or consist essentially of an N3-substituted-2-methoxy-6-amino-4-pyrimidone and an amine salt.

When the amine compound is a free amine, step (c) can include adding a salt of that amine to the reaction mixture. Thus, the reaction mixture can include or consist essentially of an N3-substituted-2-methoxy-6-amino-4-pyrimidone, an amine salt, and the corresponding free amine. The amine salt can be an aryl amine salt, e.g., an optionally substituted aniline salt, a 3,4-disubstituted aniline salt, or a salt of 3-methyl-4-ethyl aniline. Alternatively, the amine salt can be a benzylamine salt, e.g., an optionally substituted benzylamine salt, a 3,4-disubstituted benzylamine salt, or a salt of 3,4-dichlorobenzylamine salt.

An “alkyl” is a branched or unbranched hydrocarbon that may be substituted or unsubstituted. Alkyl groups for the purpose of the invention can have 1-10 carbon atoms; for example, an alkyl group can have 1-6, 2-10, 3-8, 1-5, or 2-6 carbon atoms. Examples of branched alkyl groups include isopropyl, sec-butyl, isobutyl, tert-butyl, sec-pentyl, isopentyl, tert-pentyl, and isohexyl. Substituted alkyl groups may have one, two, three or more substituents, which may be the same or different, each replacing a hydrogen atom. This definition shall apply when the term “alk” is used as a prefix, e.g., “alkoxy,” or when “alkyl” is used as part of another term, e.g., “arylalkyl.”

By “substituted” is meant that one or more hydrogen atoms of a compound or portion of a compound are replaced by desired substituents, including, but not limited to, C₁₋₄ alkyl, C₁₋₆ cycloalkyl, hydroxyl, C₁₋₄ alkoxyl, amino, carboxyl, halo, cyano, azido, C₆₋₁₂ aryl, C₇₋₂₀ arylalkyl, C₄₋₆ heteroaryl, (CO)—C₁₋₆ alkyl, (CO)—C₁₋₆ aryl, (SO₂)—C₁₋₆ alkyl, (SO₃)—C₁₋₆ alkyl, (SO₂)—C₆₋₁₂ aryl, (SO₃)—C₆₋₁₂ aryl, (SO₂)—C₄₋₁₂ heteroaryl, (SO₃)—C₄₋₁₂ heteroaryl.

By “appropriately substituted” is meant that a starting material has a substitution pattern that corresponds to the substitution pattern desired in the final product. For example, if the desired product is an N3-alkyl-pyrimidone in which the N3-alkyl substituent is substituted with a hydroxy group, an alkylating agent used to prepare such a compound would be one in which the alkyl moiety is substituted with a hydroxy group.

A “non-aqueous base” is a base that can be used in a non-aqueous medium. Examples include sodium hydride, potassium carbonate, calcium hydride, cesium carbonate, sodium carbonate, and sodium bicarbonate.

“Pharmaceutically acceptable salts” are those salts derived from pharmaceutically acceptable inorganic and organic acids and bases. Examples of suitable acids include hydrochloric, hydrobromic, sulphuric, nitric, perchloric, fumaric, maleic, phosphoric, glycolic, lactic, salicylic, succinic, toluene-p-sulphonic, tartaric, acetic, citric, methanesulphonic, formic, benzoic, malonic, naphthalene-2-sulphonic and benzenesulphonic acids. Other acids such as oxalic, while not in themselves pharmaceutically acceptable, may be useful as intermediates in obtaining the compounds of the invention and their pharmaceutically acceptable acid addition salts. Salts derived from appropriate bases include alkali metal (e.g. sodium), alkaline earth metal (e.g. magnesium), ammonium, and NR₄⁺ (where R is C₁₋₄ alkyl) salts. Useful salts include hydrochlorides, hydrobromides, sulfates, mesylates, maleates, and fumarates. References hereinafter to compounds according to the invention include compounds of the general formulae shown, as well as their pharmaceutically acceptable salts.

An “amine compound” is a free amine or an amine salt, as defined below.

A “free amine” is a compound in which a nitrogen atom is covalently bonded to three groups, selected from H, optionally substituted alkyl, optionally substituted aryl, and optionally substituted arylalkyl groups. The free amines used in the reactions described herein have one or two hydrogen atoms covalently bonded to the nitrogen atom.

An “amine salt” is an acid addition salt corresponding to a free amine. Useful salts for this purpose include without limitation the hydrochloride, hydrobromide, hydroiodide, sulfate, phosphate, mesylate, maleate, fumarate, acetate, and trifluoroacetate salts of a free amine.

An “elevated temperature” is meant a temperature above room temperature, e.g., a temperature above 50° C., 75° C., 100° C., 125° C., or 150° C.

In the case where protecting groups are used in the new methods, such protecting groups can be selected from known chemical moieties recognized in the art to protect an otherwise reactive moiety against undesirable reaction during one or more particular synthetic procedures and that is selectively removable under a given set of reaction conditions. Protecting groups may be removed by standard methods after the contemplated reaction has been completed. Protecting groups and their uses are further described in T. W. Greene and P. G. M. Wuts, Protective Groups in Organic Synthesis, 2nd ed., John Wiley & Sons, New York, 1991, which is hereby incorporated by reference in its entirety.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying structures and the description below. Other features, objects, and advantages of the invention will be apparent from the description, the chemical formulas and structures, and from the claims.

### DETAILED DESCRIPTION

The invention features novel methods for synthesizing substituted pyrimidines, for example, N3-(substituted-alkyl)-6-(substituted-amino)uracils, in high yields. The novel methods include two types of transformations: the first transformation is exemplified by alkylation of 2-methoxy-6-amino-4-pyrimidone; and the second transformation is exemplified by a fusion reaction between the N3-alkylated 2-methoxy-6-amino-4-pyrimidone and an amine salt at an elevated temperature.

An example of the first transformation is shown below in Scheme 1.

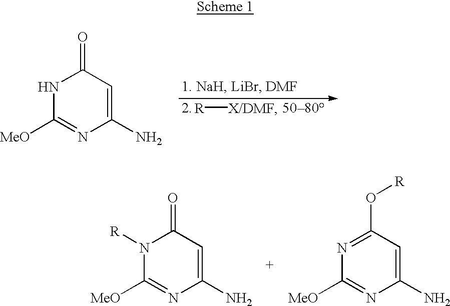

As shown in Scheme 1, when 6-amino-2-methoxy-4-pyrimidone is alkylated, the N at the 3-position, or the O at the 4-position can be alkylated. When the reaction shown in Scheme 1 is run, the major product is the N3-isomer, rather than the O4-isomer.

An exemplary reaction takes place as follows. Sodium hydride (1.2 equivalents, or eq) is added to a mixture of 6-amino-2-methoxy-4-pyrimidone (1 eq) in N,N-dimethylformamide (DMF) at 0° C. Lithium bromide (1.2-2.0 eq) is added, and the mixture is stirred for 1 hour at room temperature. This mixture is added dropwise to a solution of the alkylating agent (1.5 eq) in DMF at 50-80° C., and the reaction mixture is stirred at 50-80° C. for 3-10 hours. After cooling to room temperature, the solvent is removed to give a mixture of the N3-alkyl compound, 6-amino-2-methoxy-3-substituted-4-pyrimidone, and the O4-alkyl compound. The mixture is purified by chromatography on silica gel with chloroform:methanol as eluent. Generally, the O4 isomer is eluted first, followed by the N3 isomer.

The reaction conditions shown in Scheme 1 and described in the preceding paragraph can be modified. For example, instead of sodium hydride, other non-aqueous bases can be used. A slight excess of the base is used. For example, at least about 1.2 equivalents, at least about 1.5 equivalents, or at least about 2.0 equivalents are used.

In addition, the lithium bromide, which catalyzes the reaction, can be replaced with another alkali metal halide; alternatively, the lithium bromide or other alkali metal halide is omitted from the reaction mixture. An example of another alkali metal halide that can be used is sodium iodide. If an alkali metal halide is used, a slight excess is typically used. For example, at least about 1.2 equivalents, at least about 1.5 equivalents, or at least about 2.0 equivalents of the alkali metal halide are used.

A suitable solvent is any dipolar aprotic solvent. Examples of specific solvents include N,N-dimethylformamide, as shown in Scheme 1, as well as N,N-dimethylacetamide, dimethylsulfoxide, acetonitrile, and tetramethylenesulfone. Other dipolar aprotic solvents are also known to those of ordinary skill in the art.

The alkylating agent, R—X, includes an alkyl portion, R, and a leaving group, X. The alkyl portion may be optionally substituted with desired substituents and/or functional groups, as long as those substituents and functional groups are appropriately protected or compatible with the alkylation reaction. The leaving group can be a halide, e.g., a chloride, bromide or iodide, or another suitable leaving group, such as a mesylate, triflate, tosylate, or any other leaving group suitable for nucleophilic displacement reactions.

The starting pyrimidone, the alkali metal hydride and solvent (optionally, with an alkali metal halide as a catalyst) are typically stirred together at a temperature below room temperature, for example, about 0° C. An example of a temperature range for this reaction is approximately 0-20° C. The alkali metal halide may be added immediately, or after a period of about 1-120 minutes. After the alkali metal halide is added, the reaction may be warmed to room temperature.

This mixture is then added to a solution of the alkylating agent, R—X. Generally, the solvent used to dissolve the alkylating agent is the same solvent as in the previous step. The mixture can be added slowly, for example, it can be added dropwise. Alternatively, the mixture can be added more quickly, as the reaction conditions allow.

The resulting reaction mixture is then stirred at an elevated temperature, i.e., a temperature between room temperature and the boiling point of the solvent. An exemplary range of temperatures is 50-80° C. For example, the reaction temperature can be about 50° C. to about 60° C., or about 70° C. to about 80° C. The reaction time depends on the reactants and the reaction temperature. Generally, the reaction is complete in 3-10 hours, e.g., 3-5 hours, 6-8 hours, or 8-10 hours.

When the reaction is complete, the mixture is cooled to room temperature, and the solvent is evaporated. The crude product can be purified by chromatography on silica gel to separate the N3-substituted isomer from the O4-substituted isomer.

The invention also features a second transformation, an example of which is shown below in Scheme 2.

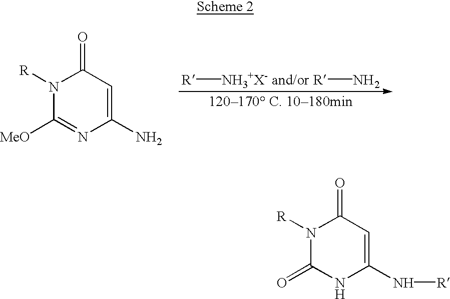

where R is as defined above, R′ is alkyl, arylalkyl, aryl, or heteroaryl, and X is an anion, such as halide (e.g., chloride). The alkyl, arylalkyl, aryl, or heteroaryl portion of R′—NH₂ can be substituted with additional functional groups as desired.

An exemplary reaction takes place as follows. A mixture of 6-amino-2-methoxy-3-substituted-4-pyrimidone (1.0 eq), a substituted-amine salt (1.2-2.5 eq), and a few drops or crystals of the substituted amine (about 0.1-1 eq) are heated at 120-170° C. for between 10 minutes and 3 hours. The mixture is cooled to room temperature, water is added, and the mixture is extracted with chloroform. The combined organic layers are dried over anhydrous magnesium sulfate. The solvent is removed under reduced pressure, and the residue is purified by chromatography on silica gel with chloroform:methanol as eluent to give the target compounds, 3-alkyl-6-(substituted-amino)uracils in high yields.

As shown in Scheme 2, the reaction results in the displacement of the 6-amino group and the demethylation of the 2-methoxy group to afford the substituted uracil in a one-pot reaction.

The reaction conditions shown in Scheme 2 and described above can be modified. Less than 1.2 equivalents of the amine salt can be used, for example, only a slight excess may be used. Similarly, more than 2.5 equivalents can be used. In some cases, the amine salt is not added at all. In such cases, the reaction mixture consists essentially of the pyrimidone and a free amine. Temperatures outside the range of 120-170° C. can be used as well. For example, temperatures between 80° C. and 200° C., or even above 200° C. can be used. Examples of suitable temperatures include at least about 80° C., at least about 100° C., at least about 120° C., or at least about 160° C. The reaction times can be shorter than 10 minutes, or longer than 180 minutes. Examples of suitable reaction times include about 10 minutes to about 30 minutes, about 60 minutes to about 90 minutes, about 120 minutes to about 150 minutes, or about 180 minutes. The reaction times will depend on the temperatures used and the substituents on the pyrimidone and the amine or amine salt. For example, if the amine or the salt of the amine contains electron-withdrawing groups, the reaction may take longer.

In some cases, it is not necessary to add the free amine. In such cases, the reaction mixture consists essentially of the pyrimidone and the amine salt. Following the guidance provided herein, appropriate reaction conditions and amounts and nature of the reactants may be determined and optimized by those of ordinary skill in the art without using undue experimentation.

The reaction can be worked up, and the product can be purified using methods other than those described above. Such methods are known by one of ordinary skill in the art.

The two methods described above can be combined to prepare 6-(substituted amino)uracil. For example, a 2-alkoxy-6-amino-4-pyrimidone is combined with a non-aqueous base and an alkylating agent that includes an appropriately substituted alkyl moiety and a leaving group to yield an N3-substituted-2-alkoxy-6-amino-4-pyrimidone. The N3-substituted-2-alkoxy-6-amino-4-pyrimidone is isolated, i.e., it is separated from the excess reactants used and from the by-products of the reaction. After isolation, the N3-substituted-2-alkoxy-6-amino-4-pyrimidone is combined with an amine compound selected from the group consisting of an amine salt and a free amine, to form a reaction mixture. The reaction mixture is heated to at least 80° C. for a time sufficient for the N3-substituted-2-alkoxy-6-amino-4-pyrimidone and the amine compound to react to form the 6-(substituted amino)uracil.

The methods of this invention yield compounds that are therapeutic agents or intermediates for therapeutic agents. For example, the products of these reactions are useful as antibiotics, antiviral agents, and/or cytotoxic agents. Alternatively, these compounds can be converted to compounds such as 3,9-disubstituted xanthines (“isoparaxanthines”) using methods known to one of skill in the art. The methods can also be used to make antibacterial compounds, e.g., as described in U.S. Pat. No. 5,516,905 and in Zhi et al., (application Ser. Nos. 60/298,357, 60/298,351, 60/348,420, and 60/348,477, filed on Jun. 15, 2001, Jun. 15, 2001, Jan. 14, 2002, and Jan. 14, 2002, respectively.)

The methods of this invention offer several advantages over other synthetic methods. For example, the method described herein for preparing substituted 4-pyrimidones results in the preferential synthesis of the N3-isomer, rather than the O4-isomer. This method can be used even when the starting 4-pyrimidones contain sensitive functional groups, such as acid-labile or alkali-labile esters. The new methods thus yield greater amounts and possibly a wider variety of therapeutically useful compounds and intermediates for therapeutically useful compounds.

The method for preparing substituted 6-(substituted-amino)-4-pyrimidones from 6-amino-4-pyrimidones offers numerous advantages as well. First, the reaction conditions are compatible with otherwise reactive functional groups that may be present on the 3-substituent of the pyrimidones. Second, using 2-alkoxypyrimidones as starting materials, two important transformations take place in a one-pot reaction: the 6-aminopyrimidone and the substituted amine react to form the desired 6-substituted compound, and the alkoxy group at the 2-position is dealkylated to form an oxo group. Third, the reaction can take place in the absence of solvent, thus obviating potentially lengthy and costly work-up procedures. Fourth, because no solvents are required, the reactions can be run at high temperatures, which shortens the reaction times, often to as little as 10 minutes.

### EXAMPLES

The following examples are to be construed as merely illustrative, and do not limit the remainder of the disclosure in any way.

### Example 1

### General Method for the Preparation of 6-amino-2-methoxy-3-substituted-4-pyrimidones

Scheme 1:

Sodium hydride (1.2 eq) was added to a mixture of 6-amino-2-methoxy-4-pyrimidone (1 eq) in N,N-dimethylformamide (DMF)) at 0° C. Then lithium bromide (1.2-2.0 eq) was added, and the mixture was stirred for 1 hour at room temperature. The mixture was added dropwise to a solution of alkyl halide (1.5 eq) in DMF at 50-80° C., and the reaction mixture was stirred at 50-80° C. for 3-10 hours. After cooling to room temperature, the solvent was removed in vacuo. The residue was purified by chromatography on silica gel with chloroform/methanol as eluent to give both 6-amino-2-methoxy-4-alkoxypyrimidine and 6-amino-2-methoxy-3-substituted-4-pyrimidone.

### Example 2

### Synthesis of 6-Amino-2-methoxy-3-(3-cyanopropyl)-4-pyrimidone

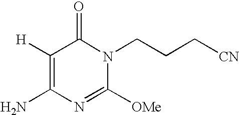

Sodium hydride (3.4 g of 60%, 85 mmol) was added to a mixture of 6-amino-2-methoxy-4-pyrimidone (10.0 g, 71 mmol) in DMF at 0° C. Then lithium bromide (8.0 g, 92 mmol) was added, and the mixture was stirred for 1 hour at room temperature. The mixture was added dropwise to a solution of 4-bromo-1-butyronitrile (15.7 g, 106 mmol) in DMF at 80° C., and the reaction mixture was stirred at 80° C. for 8 hours. After cooling to room temperature, the solvent was removed in vacuo. Water was added and the mixture was extracted with chloroform, and the organic extracts were dried over sodium sulfate. After removal of chloroform, the residue was purified by chromatography on silica gel using chloroform:methanol as eluent. After separation of 6-amino-2-methoxy-4-(3-cyanopropoxy)pyrimidine (about 30%), 6-amino-2-methoxy-3-(3-cyanopropyl)-4-pyrimidone (9.73 g, 65%) was isolated as a white solid.

300 MHz ¹H NMR (DMSO-d₆): δ 1.78 (m, 2H, CH₂), 2.50 (t, 2H, CH₂CN) 3.84 (t, 2H, CH₂N), 3.88 (s, 3H, CH₃O), 4.83 (s, 1H, C₅—H), 6.44 (s, 2H, NH₂) ppm.

### Example 3

### Synthesis of 6-Amino-2-methoxy-3-(4-acetoxybutyl)pyrimidin-4(3H)-one

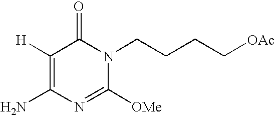

Sodium hydride (1.2 eq) was added to a mixture of 6-amino-2-methoxy-4-pyrimidone (1 eq) in DMF at 0° C. Then lithium bromide (1.5 eq) was added to the mixture and stirred for 1 hour at room temperature. The mixture was added dropwise to a solution of 4-bromo-1acetoxybutane (1.5 eq) in DMF at 50° C. Workup and chromatography gave the O4-isomer (35% yield) and 6-amino-2-methoxy-3-(4-acetoxybutyl)-4-pyrimidone (54% yield) as a white solid.

300 MHz ¹H NMR (DMSO-d₆): δ 1.52 (m, 4H, 2×CH₂), 2.0 (s, 3H, CH₃CO) 3.76 (t, 2H, CH₂O), 3.88 (s, 3H, CH₃N), ), 4.0 (t, 2H, CH₂O), 4.82 (s, 1H, C₅—H), 6.41 (s, 2H, NH2) ppm.

### Example 4

### Synthesis of 6-Amino-2-methoxy-3-[2-(2-bromoethoxy)ethyl]-4-pyrimidone

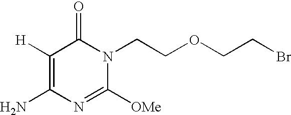

Scheme 1 was used with 9.3 g of 6-amino-2-methoxy-4-pyrimidone and 1.3 eq of bis(2-bromoethyl)ether, yielding 35% of the title compound. The O4 isomer was obtained in 20% yield.

### Example 5

### General Method for the Preparation of 3-alkyl-6-(substituted-amino)uracils

Scheme 2:

A stirred mixture of 6-amino-2-methoxy-3-substituted-4-pyrimidone (1.0 eq), amine hydrochloride (1.1-1.5 eq), and a few drops of the amine base were heated at 120-170° C. for 10 minutes to 3 hours. After cooling to room temperature, the residue was either dissolved in chloroform:methanol, or water was added and the mixture extracted with chloroform. The combined organic layers were dried over anhydrous magnesium sulfate. The solvent was removed under reduced pressure, and the residue was purified by chromatography on silica gel with chloroform:methanol as eluent, to give target compounds.

### Example 6

### Synthesis of 3-(4-Acetoxybutyl)-6-(3-ethyl-4-methylanilino)uracil

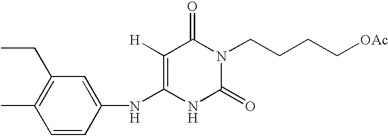

A stirred mixture of 3-(4-acetoxybutyl)-6-amino-2-methoxy-4-pyrimidone (15 g. 59 mmol), 3-ethyl-4-methylaniline hydrochloride (12.1 g, 75 mmol) and 3-ethyl-4-methylaniline (4.0 g, 29 mmol) was heated in an oil bath at 160° C. for 15 minutes. After cooling to room temperature, the residue was dissolved in chloroform:methanol (1:1), and the solution was evaporated with silica gel. The material was placed atop a silica gel column and eluted with chloroform:methanol (100% to 96% chloroform) to give crude product. Trituration with acetone:diethyl ether (1:1) gave colorless crystals of product (17.8 g, 84%). 300 MHz ¹H NMR (DMSO-d₆): δ 1.14 (t, 3H, CH₃CH₂Ar), 1.53 (m, 4H, 2×CH₂), 2.0 (s, 3H, CH₃CO), 2.24 (s, 3H, CH₃Ar), 2.57 (q, 2H, CH₂Ar), 3.71 (t, 2H, CH₂O),3.99 (t, 2H, CH₂N), 4.73 (s, 1H, C₅—H), 6.92-7.15 (m, 3H, Ar—H), 8.12 (s, 1H, NH), 10.43 (s, 1H, NH) ppm.

The product was converted to 3-(4-Hydroxybutyl)-6-(3-ethyl-4-methylanilino)uracil (shown below) as follows.

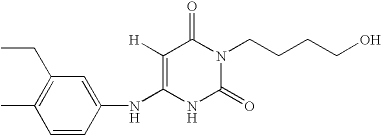

Aqueous concentrated ammonia (150 ml) was added to a stirred suspension of 3-(4-acetoxybutyl)-6-(3-ethyl-4-methylanilino)uracil, (10.5 g, 24 mmol) in 150 ml of methanol at room temperature. After 30 minutes, all solid dissolved, and the solution was stirred for 72 hours. The solvent was removed, and the solid was coevaporated three times with methanol, and filtered from methanol to give the product as a colorless solid (9.0 g, 97%).

This product, the hydroxy compound, was further converted to 3-(4-Iodobutyl)-6-(3-ethyl-4-methylanilino)uracil (shown below) as follows.

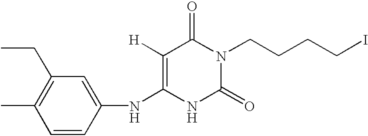

Trimethylsilyl iodide (19.4 g, 47 mmol) was added to a stirred solution of 3-(4-hydroxybutyl)-6-(3-ethyl-4-methylanilino)uracil, (7.7 g, 24.3 mmol)) in dry chloroform (300 ml). The reaction mixture was stirred at reflux for 12 hours, until disappearance of starting material (TLC). A saturated solution of aqueous sodium sulfite was added to decolorize the brown-purple solution. After separation of layers, the aqueous solution was extracted with chloroform, and the combined organic extracts were reduced to about one fourth volume. The solid was filtered and washed with water and acetone to give 9.9 g (95%) of the product.

300 MHz ¹H NMR (DMSO-d₆): δ 1.14 (t, 3H, CH₃CH₂Ar), 1.54-1.78 (m, 4H, 2×CH₂), 2.24 (s, 3H, CH₃Ar), 2.57 (q, 2H, CH₂Ar), 3.29 (t, 2H, CH₂I), 3.72 (t, 2H, CH₂N), 4.73 (s, 1H, C₅—H), 6.92-7.15 (m, 3H, Ar—H), 8.15 (s, 1H, NH), 10.45 (s, 1H, NH) ppm.

### Example 7

### Synthesis of 3-[2-(2-Benzyloxyethoxy)ethyl]-6-(3-ethyl-4-methylanilino)uracil

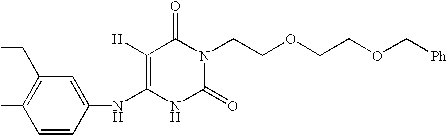

A mixture of 6-amino-3-[2-(2-benzyloxyethoxy)ethyl]-2-methoxy-4-pyrimidone (430 mg, 1.35 mmol), 3-ethyl-4-methylaniline hydrochloride (254 mg, 1.48 mmol) and a few drops of 3-ethyl-4-methylaniline was heated at 160° C. for 3 hours. After cooling to room temperature, water (15 ml) was added, and the mixture was extracted with chloroform (3×40 ml). The combined organic layers were dried over anhydrous magnesium sulfate. The solvent was removed under reduced pressure, and the residue was purified by chromatography on silica gel with chloroform:methanol (99:1-97:3) as eluent, to give 410 mg (72% yield) of 3-[2-(2-benzyloxyethoxy)ethyl]-6-(3-ethyl-4-methylanilino)uracil. Crystallization from ethanol gave white crystals.

300 MHz ¹H NMR (DMSO-d₆): δ 1.13 (t, 3H, CH₃CH₂), 2.24 (s, 3H, CH₃Ar), 2.57 (q, 2H, CH₂Ar), 3.53 (m, 6H, 3×CH₂), 3.88 (t, 2H, CH₂N), 4.47 (s, 2H, PhCH₂), 4.72 (s, 1H, C₅—H), 6.92-7.15 (m, 3H, Ar—H), 7.25-7.36 (m, 5H, Ph—H), 8.16 (s, 1H, NH), 10.49 (s, 1H, NH) ppm.

### Example 8

### Synthesis of 3-(4-Ethoxycarbonylbutyl)-6-(3-ethyl-4-methylanilino)uracil

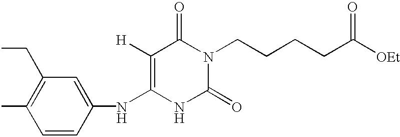

A mixture of 6-amino-3-[4-(ethoxycarbonyl)butyl]-2-methoxy-4-pyrimidone (608 mg, 2.26 mmol), 3-ethyl-4-methylaniline hydrochloride (430 mg, 2.50 mmol) and a few drops of 3-ethyl-4-methylaniline was heated at 160° C. for 3 hours. Workup gave 632 mg (75% yield) of 3-[4-(ethoxycarbonyl)butyl]-6-(3-ethyl-4-methylanilino)uracil. Crystallization from ethanol gave white crystals.

300 MHz ¹H NMR (DMSO-d₆): δ 1.11-1.19 (m, 6H, 2×CH₃), 1.49 (m, 4H, 2×CH₂), 2.24 (s, 3H, CH₃Ar), 2.30 (t, 2H, CH₂CO₂Et), 2.57 (q, 2H, CH₂Ar), 3.69 (t, 2H, CH₂N), 4.04 (q, 2H, CO₂CH₂), 4.72 (s, 1H, C₅—H), 6.92-7.15 (m, 3H, Ar—H), 8.16 (s, 1H, NH), 10.48 (s, 1H, NH) ppm.

The product was further converted to 3-(5-Hydroxypentyl)-6-(3-ethyl-4-methylanilino)uracil (shown below) as follows.

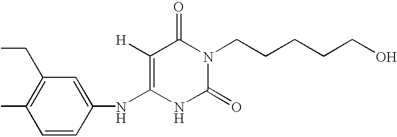

A solution of 1.0 M lithium aluminum hydride in tetrahydrofuran (1.5 ml) was added dropwise to a stirred solution of 3-[4-(ethoxycarbonyl)butyl]-6-(3-ethyl-4-methylanilino)uracil, (160 mg, 0.43 mmol) in anhydrous tetrahydrofuran (30 ml) at room temperature. The reaction mixture was stirred at room temperature until disappearance of the stating material (20 minutes). Methanol (5 ml) was added dropwise to the solution, and the solvents were removed. Ethanol was added, the mixture was filtered, and the solid washed carefully with ethanol. The solvent was removed, and the residue was purified by chromatography on silica gel with chloroform:methanol (98:2-96:4) as eluent, to give 141 mg (99% yield) of 3-(5-hydroxypentyl)-6-(3-ethyl-4-methylanilino)uracil. Crystallization from ethanol:water (1:1) gave white crystals.

300 MHz ¹H NMR (DMSO-d₆): δ 1.14 (t, 3H, CH₃CH₂Ar), 1.20-1.30 (m, 2H, CH₂), 1.37-1.52 (m, 4H, 2×CH₂), 2.24 (s, 3H, CH₃Ar), 2.57 (q, 2H, CH₂Ar), 3.34 (t, 2H, CH₂O), 3.67 (t, 2H, CH₂N), 4.35 (t, 1H, OH), 4.72 (s, 1H, C₅—H), 6.92-7.15 (m, 3H, Ar—H), 8.18 (s, 1H, NH), 10.48 (s, 1H, NH) ppm.

### Example 9

### Synthesis of 3-[(N,N-Diethylaminocarbonyl)methyl]-6-(3-ethyl-4-methylanilino)uracil

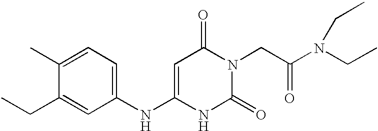

Scheme 2 gave the product in 59% yield. 300 MHz ¹H NMR (DMSO-d₆): δ 10.53 (s, 1H, NH), 8.19 (S, 1H, NH), 6.94-7.15 (m, 3H, Ar—H), 4.72 (s, 1H, C₅—H), 4.49 (s, 2H, NCH₂), 3.26 (m, 4H, N(CH₂)₂), 2.59 (q, 2H, ArCH₂), 2.24 (s, 3H, ArCH₃), 0.98-1.17 (m, 9H, 3×CH₃) ppm.

### Example 10

### Synthesis of 3-[2-(2-Methoxyethoxy)ethyl]-6-(3-ethyl-4-methylanilino)uracil

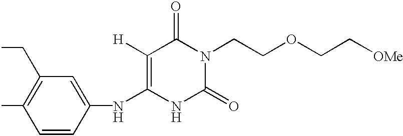

Scheme 2 gave the product in 74% yield. 300 MHz ¹H NMR (DMSO-d₆): δ 1.13 (t, 3H, CH₃CH₂), 2.24 (s, 3H, CH₃Ar), 2.57 (q, 2H, CH₂Ar), 3.22 (s, 3H, CH₃O), 3.37 (m, 2H, CH₂O), 3.49 (m, 4H, 2×CH₂O), 3.85 (t, 3H, CH₂N), 4.70 (s, 1H, C₅—H), 6.92-7.15 (m, 3H, Ar—H), 8.12 (s, 1H, NH), 10.40 (s, 1H, NH) ppm.

### Example 11

### Synthesis of 3-[2-(N,N-diethylamino)ethyl]-6-(3-ethyl-4-methylanilino uracil

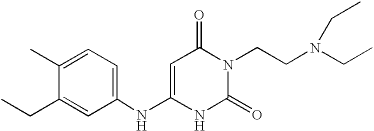

Scheme 2 gave the product in 61% yield. 300 MHz ¹H NMR (DMSO-d₆): 10.34 (s, 1H, NH), 8.13 (S, 1H, NH), 6.87-7.16 (m, 3H, Ar—H), 4.72 (s, 1H, C₅—H), 3.77 (t, 2H, NCH₂), 3.22 (m, 2H, CH₂N), 2.58 (q, 2H, ArCH₂), 2.43 (m, 4H, 2×NCH₂), 2.20 (s, 3H, ArCH₃), 1.16 (t, 3H, ArCH₂CH₃), 1.00 (m, 6H, 2×CH₃) ppm.

### Example 12

### Synthesis of 3-[2-(Methanesulfonylamino)ethyl]-6-(3-ethyl-4-methylanilino)uracil

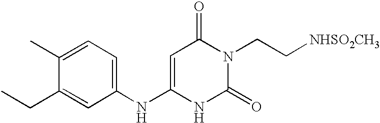

Scheme 2 gave the product in 92% yield. 300 MHz ¹H NMR (DMSO-d₆): 10.53 (s, 1H, NH), 8.18 (S, 1H, NH), 7.16 (S, 1H, NH), 6.90-7.13 (m, 3H, Ar—H), 4.73 (s, 1H, C₅—H), 3.83 (t, 2H, NCH₂), 3.09 (m, 2H, CH₂NH), 2.88 (s, 3H, SO₂CH₃), 2.59 (q, 2H, ArCH₂), 2.20 (s, 3H, ArCH₃), 1.14 (t, 3H, ArCH₂CH₃)ppm.

### Example 13

### Synthesis of 3-[2-(N-morpholino)ethyl]-6-(3-ethyl-4-methylanilino)uracil

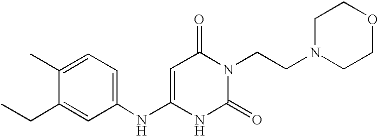

Scheme 2 gave the product in 75% yield. 300 MHz ¹H NMR (DMSO-d₆): 10.48 (s, 1H, NH), 8.14 (S, 1H, NH), 6.90-7.18 (m, 3H, Ar—H), 4.72 (s, 1H, C₅—H), 3.82 (m, 2H, NCH₂), 3.47 (m, 4H, CH₂OCH₂), 3.24 (m, 2H, CH₂N), 2.58 (q, 2H, ArCH₂), 2.40 (m, 4H, CH₂NCH₂), 2.23 (s, 3H, ArCH₃), 1.14 (t, 3H, ArCH₂CH₃)ppm.

### Example 14

### Synthesis of 3-(8-Hydroxyoctyl)-6-(3-ethyl-4-methylanilino)uracil

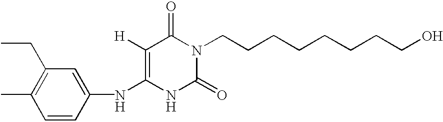

Scheme 2 gave the product in 78% yield. 300 MHz ¹H NMR (DMSO-d₆): δ 1.14 (t, 3H, CH₃CH₂Ar), 1.20-1.30 (m, 8H, 4×CH₂), 1.37-1.52 (m, 4H, 2×CH₂), 2.21 (s, 3H, CH₃Ar), 2.57 (q, 2H, CH₂Ar), 3.35 (m, 2H, CH₂O), 3.64 (t, 2H, CH₂N), 4.30 (t, 1H, OH), 4.69 (s, 1H, C₅—H), 6.92-7.15 (m, 3H, Ar—H), 8.05 (s, 1H, NH), 10.35 (s, 1H, NH) ppm.

### Example 15

### Synthesis of 3-(3-Cyanopropyl)-6-(3-ethyl-4-methylanilino)uracil

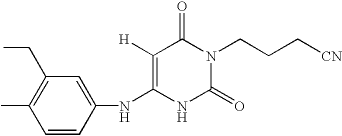

Scheme 2 gave the product in 81% yield. 300 MHz ¹H NMR (DMSO-d₆): δ 1.14 (t, 3H, CH₃CH₂Ar), 1.79 (m, 2H, CH₂), 2.24 (s, 3H, CH₃Ar), 2.50 (t, 2H, CH₂CN), 2.57 (q, 2H, CH₂Ar), 3.79 (t, 2H, CH₂N), 4.74 (s, 1H, C₅—H), 6.92-7.15 (m, 3H, Ar—H), 8.12 (s, 1H, NH), 10.47 (s, 1H, NH) ppm.

The product was further converted to 3-(4-Aminobutyl)-6-(3-ethyl-4-methylanilino)uracil hydrochloride (shown below) as follows.

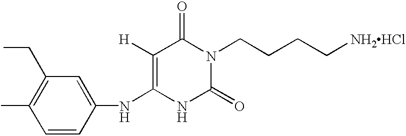

Step 1. A solution of 0.5 M lithium aluminum hydride in diglyme (3 eq) was added dropwise to a stirred solution of 3-(3-cyanopropyl)-6-(3-ethyl-4-methylanilino)uracil, (1 eq) in anhydrous diglyme at room temperature. The reaction mixture was stirred at room temperature until disappearance of the starting material. Methanol was added dropwise to the solution, and the solvents were removed. Ethanol was added and the mixture filtered, and the solid was washed carefully with ethanol. Ethanol was removed, and the residue was purified by chromatography on silica gel with chloroform:methanol as eluent, to give 3-(4-aminobutyl)-6-(3-ethyl-4-methylanilino)uracil (91% yield).

Step 2. 3-(4-Aminobutyl)-6-(3-ethyl-4-methylanilino)uracil was dissolved in chloroform and methanol, and a solution of 4.0 M hydrogen chloride in dioxane was added. The mixture was stirred at room temperature for 1 hour. The solvents were removed to give 3-(4-aminobutyl)-6-(3-ethyl-4-methylanilino)uracil hydrochloride as a white solid.

300 MHz ¹H NMR (DMSO-d₆): δ 1.11 (t, 3H, CH₃CH₂Ar), 1.50 (m, 4H, 2×CH₂), 2.21 (s, 3H, CH₃Ar), 2.57 (q, 2H, CH₂Ar), 2.78 (m, 2H, CH₂NH₂), 3.72 (t, 2H, CH₂N), 4.75 (s, 1H, C₅—H), 6.92-7.15 (m, 3H, Ar—H), 7.86 (br, 3H, NH₃), 8.89 (s, 1H, NH), 10.76 (s, 1H, NH) ppm.

### Example 16

### Synthesis of 3-(4-Cyanobutyl)-6-(3-ethyl-4-methylanilino)uracil

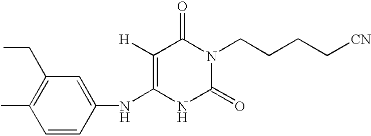

Scheme 2 gave the product in 78% yield. 300 MHz ¹H NMR (DMSO-d₆): δ 1.14 (t, 3H, CH₃CH₂Ar), 1.50-1.61 (m, 4H, 2×CH₂), 2.24 (s, 3H, CH₃Ar), 2.58 (m, 4H, CH₂CN, CH₂Ar), 3.72(t, 2H, CH₂N), 4.73 (s, 1H, C₅—H), 6.92-7.15 (m, 3H, Ar—H), 8.12 (s, 1H, NH), 10.45 (s, 1H, NH) ppm.

### Example 17

### Synthesis of 3-{2-[2-Hydroxyethoxy-(2-ethoxy)]ethyl}-6-(3-ethyl-4-methylanilino)uracil

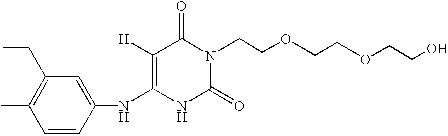

Scheme 2 gave the product in 72% yield. 300 MHz ¹H NMR (DMSO-d₆): δ 1.14 (t, 3H, CH₃CH₂), 2.24 (s, 3H, CH₃Ar), 2.57 (q, 2H, CH₂Ar), 3.32-3.50 (m, 10H, 5×CH₂O), 3.87 (t, 2H, CH₂N), 4.56 (t, 1H, OH), 4.72 (s, 1H, C₅—H), 6.92-7.16 (m, 3H, Ar—H), 8.16 (s, 1H, NH), 10.50 (s, 1H, NH) ppm.

### Example 18

### Synthesis of 3-(4,5-Dihydroxypentyl)-6-(3-ethyl-4-methylanilino)uracil

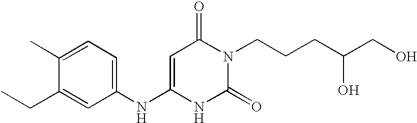

Scheme 2, starting with 6-amino-2-methoxy-3-[4,5-bis-(trimethylsilyloxy)pentyl]-4-pyrimidone, gave the product in 72% yield. 300 MHz ¹H NMR (DMSO-d₆): 10.42 (s, 1H, NH), 8.12 (S, 1H, NH), 6.90-7.13 (m, 3H, Ar—H), 4.72 (s, 1H, C₅—H), 4.41 (m, 2H, 2×OH), 3.66 (t, 2H, NCH₂), 3.20-3.36 (m, 3H, OCH₂, OCH), 2.58 (q, 2H, ArCH₂), 2.20 (s, 3H, ArCH₃), 1.33-1.68 (m, 4H, CH₂CH₂), 1.16 (t, 3H, ArCH₂CH₃) ppm.

### Example 19

### Synthesis of 3-[3-(N-Morpholino)propyl]-6-(3-ethyl-4-methylanilino)uracil

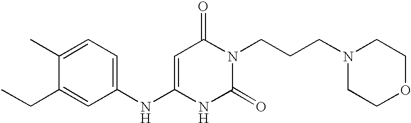

Scheme 2 gave the product in 78% yield. 300 MHz ¹H NMR (DMSO-d₆): 10.40 (s, 1H, NH), 8.12 (S, 1H, NH), 6.95-7.18 (m, 3H, Ar—H), 4.75 (s, 1H, C₅—H), 3.78 (t, 2H, NCH₂), 3.59 (m, 4H, CH₂OCH₂), 2.60 (q, 2H, ArCH₂), 2.22-2.38 (m, 6H, NCH₂×3), 2.23 (s, 3H, ArCH₃), 1.68 (m, 2H, CH₂), 1.16 (t, 3H, ArCH₂CH₃)ppm.

### Example 20

### Synthesis of 3-(3-Hydroxy-2-methylpropyl)-6-(3-ethyl-4-methylanilino)uracil

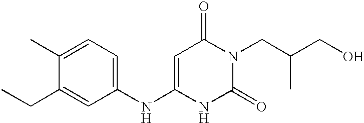

Scheme 2 gave the product in 60% yield. 300 MHz ¹H NMR (DMSO-d₆): 10.52 (s, 1H, NH), 8.19 (S, 1H, NH), 6.92-7.15 (m, 3H, Ar—H), 4.73 (s, 1H, C₅—H), 4.39 (t, 1H, OH), 3.61 (t, 2H, NCH₂), 3.33 (m, 2H, CH₂O), 2.59 (q, 2H, ArCH₂), 2.22 (s, 3H, ArCH₃), 1.96 (m, 1H, CH), 1.15 (t, 3H, ArCH₂CH₃), 0.88 (d, 3H, CH₃)ppm.

### Example 21

### 3-[2-(2-N-morpholinoethoxy)ethyl]-6-(3,4-dichlorobenzylamino)uracil

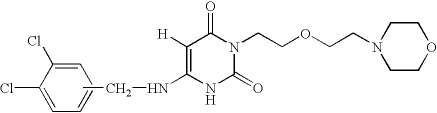

A mixture of 6-amino-2-methoxy-3-[2-(2-N-morpholinoethoxy)ethyl]-4-pyrimidone (1.63 g, 5.6 mmol), 3,4-dichlorobenzyl hydrochloride (1.42 g, 6.7 mmol) and 3,4-dichlorobenzylamine (2.8 mmol) was heated in a oil bath at 160° C. for 2 hours. Purification on a silica gel column with 10% methanol in chloroform as eluent gave the crude product. Crystallization from acetone/diethyl ether gave 1.25 g (47%) of pure product.

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

What is claimed is:

1. A method of preparing an N3-substituted-4-pyrimidone, the method comprising
(a) contacting a 4-pyrimidone with a non-aqueous base and an alkylating agent that includes an appropriately substituted alkyl moiety and a leaving group to form a reaction mixture; and
(b) maintaining the reaction mixture for a time sufficient for the 4-pyrimidone to be substituted in the N3-position with the appropriately substituted alkyl moiety.

2. The method of claim 1, wherein the 4-pyrimidone is a 2-alkoxy-6-amino-4-pyrimidone.

3. The method of claim 2, wherein the 2-alkoxy-6-amino-4-pyrimidone is a 2-methoxy-6-amino-4-pyrimidone.

4. The method of claim 1, wherein the non-aqueous base is an alkali metal hydride.

5. The method of claim 4, wherein the alkali metal hydride is sodium hydride.

6. The method of claim 4, wherein the reaction mixture further includes an alkali metal halide.

7. The method of claim 5, wherein the reaction mixture further includes an alkali metal halide.

8. The method of claim 7, wherein the alkali metal halide is lithium bromide.

9. The method of claim 1, wherein the reaction mixture includes an aprotic polar organic solvent.

10. The method of claim 9, wherein the solvent is N,N-dimethylformamide.

11. The method of claim 1, wherein a sufficient time is 1 second to 240 minutes.

12. The method according to claim 1, wherein the amine salt is selected from the group consisting of a hydrochloride, hydrobromide, hydroiodide, sulfate, phosphate, mesylate, maleate, fumarate, acetate, and trifluoroacetate salt of a free amine.

13. A method of preparing a 6-(substituted amino)uracil, the method comprising
(a) combining a 2-alkoxy-6-amino-4-pyrimidone with an amine compound selected from the group consisting of an amine salt and the corresponding free amine, to form a reaction mixture; and
(b) heating the reaction mixture to at least 80° C. for a time sufficient for the 2-alkoxy-6-amino-4-pyrimidone and the amine compound to react to form said 6-(substituted amino)uracil.

14. The method of claim 13, wherein the 6-(substituted amino)uracil is an N3-substituted-6-(substituted amino)uracil.

15. The method of claim 14, wherein the N3-substituent is an optionally substituted alkyl.

16. The method of claim 13, wherein the amine compound is a free amine.

17. The method of claim 13, wherein the amine compound is an amine salt.

18. The method of claim 17, wherein step (a) further comprises adding the corresponding free amine to the reaction mixture.

19. The method of claim 18, wherein the reaction mixture consists essentially of an N3-substituted-2-methoxy-6-amino-4-pyrimidone, the amine salt, and the corresponding free amine.

20. The method of claim 19, wherein the amine salt is an aryl amine salt.

21. The method of claim 20, wherein the aryl amine salt is an optionally substituted aniline salt.

22. The method of claim 21, wherein the aniline salt is a 3,4-disubstituted aniline salt.

23. The method of claim 22, wherein the 3,4-disubstituted aniline salt is a salt of 3-methyl-4-ethyl aniline.

24. The method of claim 20, wherein the aryl amine salt is a benzylamine salt.

25. The method of claim 24, wherein the benzylamine salt is an optionally substituted benzylamine salt.

26. The method of claim 25, wherein the benzylamine salt is a 3,4-disubstituted benzylamine salt.

27. The method of claim 26, wherein the 3,4-disubstituted benzylamine salt is a salt of 3,4-dichlorobenzylamine.

28. The method of claim 13, wherein the amine salt is selected from the group consisting of a hydrochloride, hydrobromide, hydroiodide, sulfate, phosphate, mesylate, maleate, fumarate, acetate, and trifluoroacetate salt of a free amine.

29. A method for preparing a 6-(substituted amino)uracil, the method comprising
(a) contacting a 2-alkoxy-6-amino-4-pyrimidone with a non-aqueous base and an alkylating agent that includes an appropriately substituted alkyl moiety and a leaving group, for a time sufficient to produce an N3-substituted-2-alkoxy-6-amino-4-pyrimidone;
(b) isolating the N3-substituted-2-alkoxy-6-amino-4-pyrimidone;
(c) combining the N3-substituted-2-alkoxy-6-amino-4-pyrimidone with an amine compound selected from the group consisting of an amine salt and the corresponding free amine, to form a reaction mixture; and
(d) heating the reaction mixture to at least 80° C. for a time sufficient for the N3-substituted-2-alkoxy-6-amino-4-pyrimidone and the amine compound to react to form said 6-(substituted amino)uracil.

30. The method of claim 29, wherein the 2-alkoxy-6-amino-4-pyrimidone is a 2-methoxy-6-amino-4-pyrimidone.

31. The method of claim 29, wherein the non-aqueous base is an alkali metal hydride.

32. The method of claim 31, wherein the alkali metal hydride is sodium hydride.

33. The method of claim 29, wherein step (a) is conducted in the presence of an alkali metal halide.

34. The method of claim 33, wherein the alkali metal halide is lithium bromide.

35. The method of claim 29, wherein step (a) is conducted in an aprotic polar organic solvent.

36. The method of claim 35, wherein the solvent is N,N-dimethylformamide.

37. The method of claim 29, wherein the 6-(substituted amino)uracil is an N3-substituted-6-(substituted amino)uracil.

38. The method of claim 37, wherein the N3-substituent is an optionally substituted alkyl.

39. The method of claim 29, wherein the amine compound is a free amine.

40. The method of claim 39, wherein the reaction mixture consists essentially of an N3-substituted-2-methoxy-6-amino-4-pyrimidone and a free amine.

41. The method of claim 29, wherein the amine compound is an amine salt.

42. The method of claim 41, wherein the reaction mixture consists essentially of an N3-substituted-2-methoxy-6-amino-4-pyrimidone and an amine salt.

43. The method of claim 41, wherein step (c) further comprises adding the corresponding free amine to the reaction mixture.

44. The method of claim 43, wherein the reaction mixture consists essentially of an N3-substituted-2-methoxy-6-amino-4-pyrimidone, an amine salt, and the corresponding free amine.

45. The method of claim 44, wherein the amine salt is an aryl amine salt.

46. The method of claim 45, wherein the aryl amine salt is an optionally substituted aniline salt.

47. The method of claim 46, wherein the aniline salt is a 3,4-disubstituted aniline salt.

48. The method of claim 47, wherein the 3,4-disubstituted aniline salt is a salt of 3-methyl-4-ethyl aniline.

49. The method of claim 45, wherein the aryl amine salt is a benzylamine salt.

50. The method of claim 49, wherein the benzylamine salt is an optionally substituted benzylamine salt.

51. The method of claim 50, wherein the benzylamine salt is a 3,4-disubstituted benzylamine salt.

52. The method of claim 51, wherein the 3,4-disubstituted aniline salt is a salt of 3,4-dichlorobenzylamine.

53. The method according to claim 29, wherein the amine salt is selected from the group consisting of a hydrochloride, hydrobromide, hydroiodide, sulfate, phosphate, mesylate, maleate, fumarate, acetate, and trifluoroacetate salt of a free amine.
